(19) United States
(12) Patent Application Publication
(10) Pub. No.: US 2016/0292373 A1
(43) Pub. Date: Oct. 6, 2016

(54) ADAPTIVE USER INTERFACE BASED ON HEALTH MONITORING EVENT

(71) Applicant: PREVENTICE, INC., Rochester, MN (US)

(72) Inventors: Jeffrey R. SPORS, Eyota, MN (US); Richard M. SMITH, Oronoco, MN (US)

(73) Assignee: PREVENTICE, INC., Rochester, MN (US)

(21) Appl. No.: 14/679,592

(22) Filed: Apr. 6, 2015

(51) Int. Cl. G06F 19/00; G06Q 10/06

(52) U.S. Cl. CPC G06F 19/3406; G06Q 10/0633

(57) ABSTRACT

Collecting biometric data from a patient provides numerous opportunities for a care provider to monitor the health of the patient. In one embodiment, the biometric data is used to identify health events that are processed in a workflow that includes a plurality of interconnected tasks and queues. In one embodiment, at least one task of the workflow includes displaying a user interface to the care provider. For example, a workflow for a cardiac event may include displaying an electrocardiography graph for a patient measured over the last two minutes to a trained technician who then provides an action which determines the next step in the workflow. Because the biometric data may be used to identify different types of health events (e.g., cardiac events versus respiratory events), a portal may display different graphical user interfaces to the care provider for the different types of health events.

### BACKGROUND

1. Field

Embodiments presented herein generally describe software tools related to health care. More specifically, embodiments presented herein provide techniques for displaying information related to a health event on an adaptive user interface.

2. Description of the Related Art

The Internet of Things (IoT) generally refers to the ability of many devices, aside from conventional computing platforms, to connect to computer networks. In the health care field, the ability to network virtually any devices allows a person's health to be monitored outside of a hospital or physician office. For example, network aware devices may be embedded in clothing, worn using a support device, or located in user's house. Sensors in such devices can communicate with a mobile device carried by the user (e.g., a mobile phone or tablet) or a remote system over a network. The mobile device can forward data received from the sensors to remote computing systems for processing as well as perform processing locally.

Using the data retrieved from the sensors, a physician can monitor the health of the patient remotely. For example, a physician can check in on a patient to monitor her heart rate, blood pressure, or check for changes in weight using the data collected by the sensors. The physician can then determine whether a change to the patient's treatment is needed—e.g., changing medication dosage, scheduling an additional physician visit, and the like.

### SUMMARY

The present disclosure includes a method and a computer-readable medium that contains program code that receives a health event for a patient derived from monitoring biometric data generated by one or more sensor devices. The method and program code generate a first graphical user interface (GUI) comprising a plurality of health events, where the health event is one of the plurality of health events. Upon receiving a selection of one of the plurality of health events, the method and program code generating a second GUI by identifying a type of the selected health event that determines a layout of the second GUI, wherein the plurality of health events includes health events of at least two different event types.

The present disclosure also includes a system that includes at least one sensor device to collect biometric data associated with a patient and a portal. The portal is configured to receive a health event for the patient derived from the biometric data and generate a first GUI comprising a plurality of health events, where the health event is one of the plurality of health events. Upon receiving a selection of one of the plurality of health events, the portal is also configured to generate a second GUI by identifying a type of the selected health event that determines a layout of the second GUI, where the plurality of health events includes health events of at least two different event types.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 illustrates an example computing environment, according to one embodiment.

FIG. 2 is a flow diagram illustrating executing a workflow for a health event, according to one embodiment.

FIG. 3 illustrates a workflow, according to one embodiment.

FIG. 4 is a flow diagram for displaying a user interface customized for a health event, according to one embodiment.

FIGS. 5A-5B illustrate user interfaces displaying lists of health events, according to several embodiments.

FIGS. 6A-6C illustrate user interfaces customized for different health events, according to several embodiments.

FIG. 7 illustrates a portal for displaying customized user interfaces, according to one embodiment.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Network aware devices provide a variety of opportunities for a care provider (e.g., a physician, nurse, technician, etc.) to improve patient care. An event manager can use the data provided by network aware devices or an “internet of things” (IoT) device to identify health events that range from identifying critical health care issues such as cardiac or respiratory emergencies to maintenance events where the network aware device fails, e.g., because a battery is low or a wire is disconnected. To process health related events, an event manager may process events using a collection of defined paths.

In one embodiment, a task or queue in a workflow displays a user interface (UI) to the care provider. For example, a workflow initiated following a cardiac event may display an electrocardiography (ECG) graph for a patient measured over the last two minutes to a trained technician who performs an action which determines the next step in the workflow. For instance, the event manager may have triggered a cardiac event by determining that the heart activity in the ECG graph was irregular. However, a prescreening task may determine that the heart activity is noise or is a normal rhythm, or a technician may disagree with the event manger and determine the event can be ignored. Alternatively, the technician may agree that the heart activity is irregular and escalate the cardiac event—e.g., send the event to another technician for a second opinion, forward the event to the patient's physician, or send instructions to the patient to perform a therapeutic activity.

Because the event manager may use data retrieved from the devices to identify different types of health events, a portal may display a different graphical user interface (GUI) for each different type of health event. The arrangement, as well as the content in the GUIs, may vary depending on the type of the health event. In one embodiment, the portal displays a first GUI that lists the identified health events from patients. Once a care provider selects a health event from the list, the portal displays a second GUI customized for the selected health event. For example, a GUI for a cardiac event may include an ECG and baseline charts, while a GUI for a respiratory event includes a chart tracking oxygen levels in the blood. Using the second GUI, the care provider can select a particular action to take—e.g., escalating the event, asking the patient what symptoms she has, instructing the event manager to continue to log the event, request the patient re-take a measurement, and the like. In addition to customizing the GUIs based on event type, the portal may change the GUI based on other types of information such as a priority associated with the health event, the particular care provider viewing the GUI (e.g., a physician may want to see different information than a ECG technician), whether the sensors monitoring a patient have triggered multiple health events, and the like.

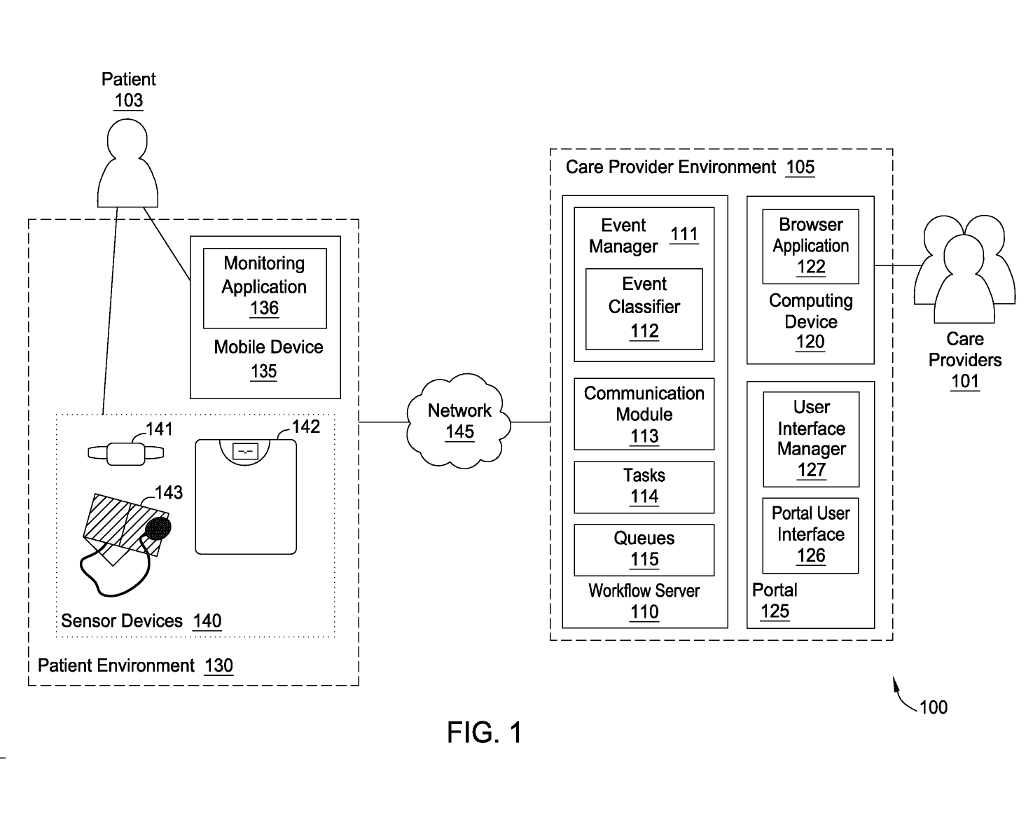

FIG. 1 illustrates an example computing environment 100, according to one embodiment. As shown, the computing environment 100 may include a care provider environment 105 and a patient environment 130, each connected to one another via a network 145. The environments 105 and 130 allow a care provider 101 (e.g., a technician, nurse, physician, etc.) to monitor biometric data generated by the patient 103.

The care provider environment 105 includes workflow server 110, a computing device 120, and a portal 125. Each of the workflow server 110, device 120, and portal 125 may be a physical computing system which includes one or more computing devices or may be a virtual computer instance (e.g., executing in a cloud computing platform). A care provider 101 may use the computing device 120 to access (e.g., via a browser application 122, a native application on device 120, etc.) a portal UI 126 hosted by the portal 125.

The workflow server 110 includes various applications and data to identify and handle health events corresponding to patient 103. As shown, workflow server 110 includes an event manager 111 and a communication module 113. The event manager 111 evaluates data received from the patient environment 130 using a set of tasks 114 (e.g., executable software code) and queues 115 that establish a workflow. The tasks 114 may be performed by the event manager 111 or by a care provider 101. The event manager 111 includes an event classifier 112 which processes the data to identify a type of health event. For example, different types of data received from the patient environment 130 may trigger different types of health events—e.g., an irregular heartbeat may trigger a cardiac event, while a signal indicated an electrode has become detached triggers a maintenance event. Each type of health event may correspond to a different workflow or path through the workflow. That is, different health events may traverse the tasks 114 and queues 115 using different paths. For example, a cardiac event may be evaluated using different tasks 114 in the server 110 than a maintenance event. Furthermore, paths used by the same health event to traverse the workflow may differ based on a variety of factors such as the severity of the health event, age of the patient 103, other symptoms exhibited by the patient 103, medication taken by the patient 103, and the like. For example, a high priority cardiac event may skip one or more tasks 114 or queues 115 and be immediately displayed to a care provider 101 using the portal UI 126.

The communication module 113 permits the workflow server 110 to receive data from the patient environment 130 and transmit data to the care providers 101. The communication module 113 may receive data from the sensor devices 140 which the event manager 111 uses to identify a health event and a corresponding path through the workflow server 110. The communication module 113 can then use portal 125 and computing device 120 to help the care providers 101 complete the workflow. Moreover, in addition to receiving information from the patient environment 130, the communication module 113 may enable the event manager 111 to transmit requests or instructions to the patient environment 130 such as asking the patient 103 if she has any symptoms or instructing the patient 103 to reattach a disconnected electrode.

In one embodiment, the workflow server 110 uses the portal UI 126 to help the care providers 101 move the heath events through the workflow. As will be discussed below, the structure and arrangement of the portal UI 126 changes according to the type of health event identified by the event classifier 112, or according to the characteristics of the care provider (e.g., whether the provider is a ECG tech or a primary care physician). To manage the visual aspects of the UI 126, the portal 125 includes a UI manager 127 which may store predefined Uls for the health events. For example, the UI manager 127 can change the appearance of the portal UI 126 depending on the type of health event currently selected by the care provider 101. The different arrangements may provide information relevant to the specific health events. For instance, a UI for performing a maintenance workflow may have information such as the length of time the event manager 110 has failed to receive data from the patient environment 130, while a UI for a cardiac event may display the current ECG chart and a list of medications taken by the patient 103.

In one embodiment, the path used by a health event to traverse the workflow may include tasks 114 performed by the event manager 111 as well as the care providers 101. For one or more tasks 114, the event manager 111 may filter or screen a health event to determine what queue to place the event, compare the event to one or more rules to determine an action to perform, store the event, or display the event on the portal UI 126. Alternatively, the workflow includes tasks 114 performed by the care provider 101. As an example, the event manager 111 may perform a filtering task where the health event is given a priority. Based on this priority, the event manager 111 places the event in corresponding queue 115 in the workflow server 110 which is accessible to the care provider 101 via the UI 126. Once the care provider 101 performs an action (e.g., confirms the classification of the event or agrees with an action suggested by the event manager 111), the remaining steps of the workflow may be performed by the event manager 111—e.g., send a notification to the patient 103, log the event in the history of the patient 103, route the event to a different care provider 101, or reclassify the event (if the care provider 101 indicated the initial classification was incorrect).

Patient environment 130 includes a mobile device 135 and sensor devices 140. The mobile device 135 includes a monitoring application 136 which permits communication between the sensor devices 140 and the care provider environment 105 via network 145. The monitoring application 136 may configure one or more sensor devices 140 (e.g., IoT devices) to monitor one or more patient biometric data as specified by a care plan. For example, the application 136 could configure logic on a heart rate monitor device worn by the patient to monitor the patient's heart rate. In turn, the monitoring application 136 can send the heart rate to the workflow server 110 which determines if a heath event is triggered and can process the event using the workflow as described above. In another embodiment, the heart rate monitor device, upon detecting that a threshold condition has been satisfied, could transmit an alert to the mobile device 135, which in turn transmits this information to the workflow server 110.

In one embodiment, the monitoring application 136 may use an output device (e.g., a display or audio system) on the mobile device 135 to provide information to the patient 103. For example, when traversing the workflow, the event manager 111 may request that the patient 103 inform the manager 111 of any symptoms she is experiencing. To get the patient's feedback, the monitoring application 136 may display a UI on the mobile device 135 which permits the patient 103 to list symptoms. Moreover, the application 136 may also display general information related to a care plan or the sensor devices 140 such as the patient's heart rate or weight, status of the sensors devices 140, etc.

In one embodiment, sensor devices 140 may interact with monitoring application 136 and assist the patient 103 in reporting patient vitals and other information to the care provider environment 105. As shown, such sensor devices 140 may include a body sensor 141, a weighing scale 142, and a blood pressure cuff 143. Each of the sensor devices 140 may capture different vitals of the patient 103. For example, when applied to the body of patient 103, the body sensor 141 captures biometric data (e.g., heart rate, ECG data, etc.) in real-time. In addition, each of the sensor devices 140 may be configured to transmit the body-related metrics electronically to the monitoring application 136 on the mobile device 135. In turn, the monitoring application 136 sends the captured metrics to the workflow server 110 which can be used to trigger health events which are processed using the tasks 114 and queues 115.

In one embodiment, upon detecting an observation threshold has been reached, the sensor devices 140 perform an initial classification of the health event. In a particular embodiment, the mobile device 135 is configured to perform the initial classification of the health event. For example, the body sensor 141, upon detecting that the ECG data collected from the patient 103 indicates an erratic heart behavior, could classify the health event as a cardiac event. This initial classification, along with the relevant ECG data (e.g., ECG data a predetermined length of time before and after the event), could be transmitted to the mobile device 135 (e.g., over a Bluetooth® communications link) where the monitoring application 136 forwards the event data on to the workflow server 110 over the network 145 (e.g., the Internet). Alternatively, instead of classifying the data, the monitoring application 136 may forward the sensor data to the event manager 111 which uses the event classifier 112 to identify health events which are then processed in the workflow.

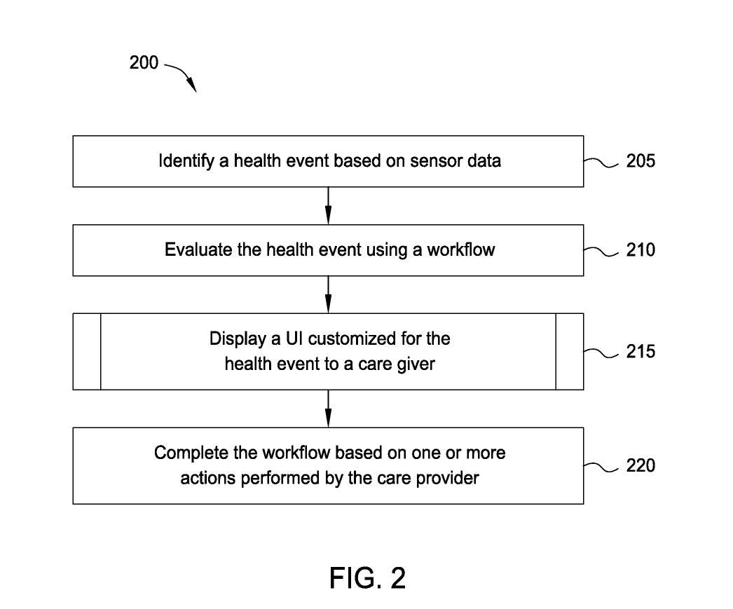

FIG. 2 is a flow diagram 200 illustrating a workflow for processing health events, according to one embodiment. At step 205, the workflow server receives data from sensor devices monitoring biometrics of a patient—e.g., the weight, heart rate, breathing rate, blood oxygen levels, status of the sensor devices, and the like. To perform one task, the event classifier evaluates the received sensor data to identify a health event—e.g., a cardiac event, respiratory event, or device maintenance event. In one embodiment, the event classifier may use a threshold or alarm to determine when the sensor data triggers a health event processed using the workflow—e.g., if the heart rate or breathing rate exceed predefined threshold for a certain period of time. Or the event classifier may use a timer to trigger a health event—e.g., the classifier triggers a health event each time five minutes worth of biometric data is received from a sensor device. However, instead of the event classifier triggering a health event, in other embodiments, a device in the patient environment such as the sensor devices or the mobile device may have initially classified the data as a health event.

At step 210, the event manager uses the interconnected tasks and queues in the workflow to process the health event. In one embodiment, the data received from the sensor devices at the workflow server may be classified in order to identify a health event if this was not done in the previous step. That is, instead of the sensor data being classified at step 205, the sensor data may be sent to the workflow server which then determines whether to trigger a health event. As described above, the path used by a health event through the workflow may differ depending on the type of the health event. For example, the workflow may have tasks for processing cardiac health events which are different from tasks for processing respiratory health events. Moreover, the same events may use different paths to traverse the workflow. For instance, the cardiac events for Patient A and Patient B may be processed at the same task. If the cardiac event for Patient A does not meet a threshold related to heart beat irregularity, the event manager may forward the event to a task that stores the cardiac event. However, if the cardiac event for Patient B meets the threshold, the event manager may forward this event to a queue that requires a care provider (e.g., an ECG technician) to evaluate the event. Thus, the same type of health events may traverse the tasks and queues in the workflow using different paths. Moreover, the queues may aggregate the events by type, technician specialty or based on the patient who triggered the event. As the health events traverse the workflow, some of the tasks may be automated (i.e., performed without human intervention) while others requires the patient or care provider to perform a task. For example, one task may send a request to a user device that instructs the patient to re-take a measurement.

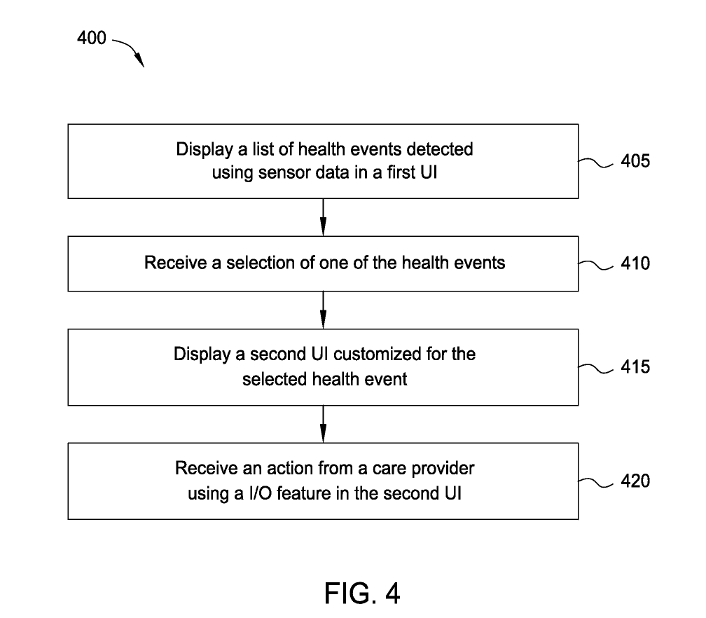

At step 215, the health event reaches a task or queue in the workflow that requests a care provider to perform an action. As such, the event manager may forward the information in the health event to the portal which then displays this information to the care provider in a UI customized for the health event. When generating the UI, the UI manager in the portal may consider the type of the health event. For example, the Uls for cardiac events, respiratory events, and maintenance events may have different arrangements or display different types of information. Furthermore, the information displayed in the UI may differ depending on the care provider viewing the health event. For example, a physician viewing a UI for a cardiac event may see a list of medications currently prescribed to the patient. However, the UI manager may omit the list of medication when the same cardiac event is viewed by an ECG technician. Further discussion of customizing a UI is provided below when describing the flow diagram in FIG. 4.

At step 220, the event manager completes the workflow based on one or more actions performed by the care provider. To determine what action to perform, the care provider uses the information displayed in the UI at step 215 such as an ECG graph, breathing rate, symptoms reported by the patient, change in weight, and the like. For example, the event manager may have determined that the ECG graph indicates that the patient's heart beat is irregular. The care provider can evaluate the same portion of the ECG graph and agree or disagree with the diagnosis reached by the event manager—i.e., agree or disagree that the patient's heart beat is irregular. In another example, the event manager may not have determined an initial diagnosis and instead relies on the care provider to provide instructions. For example, the health event may have been triggered because the breathing rate of the patient has exceeded a threshold rate for a predefined period of time. The care provider can evaluate the information corresponding to the health event and determine what action to perform—e.g., ignoring the event (if the person is just exercising), getting a second opinion for another care provider, asking the patient what symptoms she is experiencing, or forwarding the event to the patient's physician.

In one embodiment, the UI includes one or more input/output (I/O) features (e.g., buttons, text fields, chat boxes, text messaging, etc.) that allow the care provider to perform an action. For example, if the care provider determines the health event should be forwarded to her supervisor, the UI may include a button which instructs the event manager to place the health event in a queue for the supervisor. Or the care provider can use a text messaging feature in the UI to send a text message to the patient to ask the patient what symptoms she is experiencing.

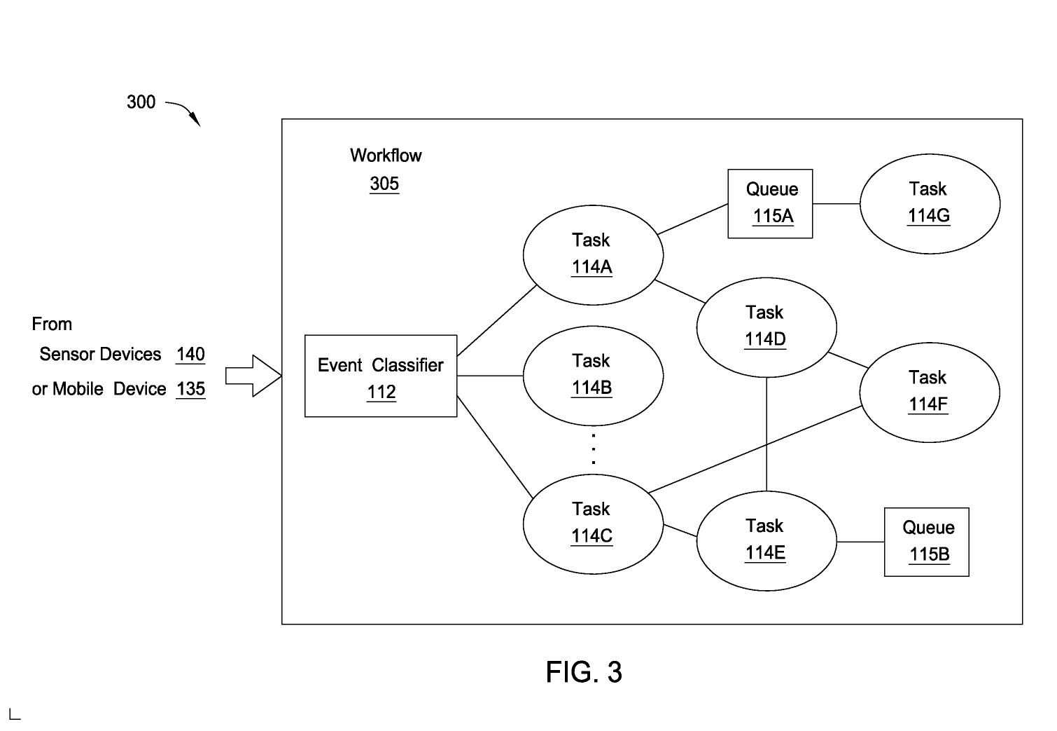

FIG. 3 illustrates an example of a workflow 305. In one embodiment, the action performed by care provider determines how the health event is processed in the workflow 305. That is, the event manager may send the event to different tasks 114 or queues 115 depending on the action performed by the care provider. For example, the event manager may send the health event to different tasks 114 depending on whether the care provider instructed the event manager to store the event, escalate the event, delay processing the event for a predefined time period, or request feedback from the patient. For instance, task 114D may require input from a care provider. Based on the action selected by the provider, the event manager may forward the event to either task 114F or 114E for further evaluation. As such, the workflow 305 uses an action performed by the care provider to determine the path traversed by the health event in the workflow 305.

Eventually, the event traverses the workflow 305 and reaches a termination task—e.g., task 114G, 114F, and Queue 115B. As described above, the path traveled by the health event through the workflow 305 may differ based on factors such as the type or priority of the event, age/condition of the patient, other actions performed by care providers, and the like.

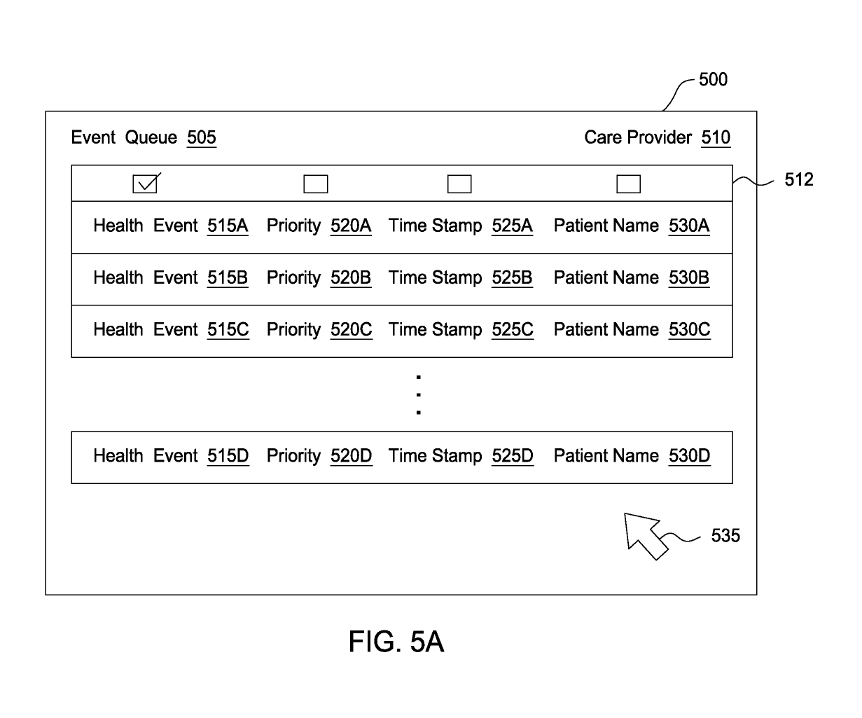

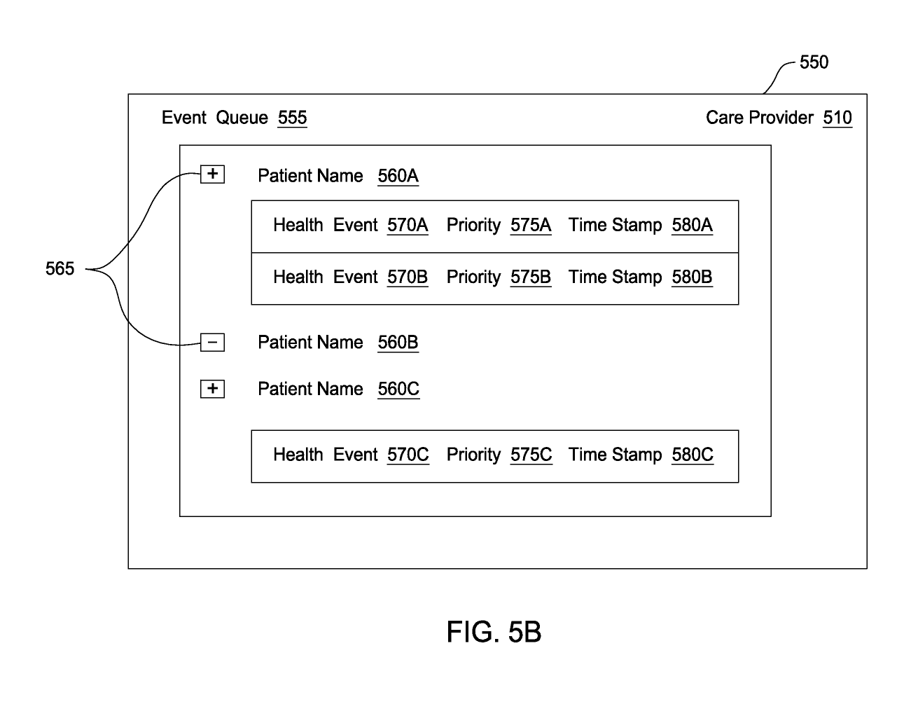

FIG. 4 is a flow diagram 400 for displaying a user interface customized for a health event, according to one embodiment. Note that flow diagram 400 generally corresponds to step 215 of flow diagram 200. At step 405, the UI manager displays a first UI to a care provider that includes a list of health events detected using sensor data. In one embodiment, the first UI is not shown to the patient but rather to one or more care providers who are trained to evaluate the biometric data retrieved from the sensor devices or troubleshoot technical problems with the sensors. Examples of such Uls are shown in FIGS. 5A-5B. As shown in FIG. 5A, GUI 500 includes an event queue 505 containing a set of health events 515. The health events may correspond to the same type of health events 515 (e.g., all cardiac events) or different health events. For example, GUI 500 may be viewed by a care provider 510 who specializes in reading and interpreting cardiac events. As such, the UI manager may display only cardiac events in the GUI 500. However, if GUI 500 is intended for a family physician who treats patients with varying ailments, the event queue 505 may include health events 515 from all her patients which may include different types of events—e.g., respiratory, cardiac, weight change, etc. Thus, the layout and information provided by GUI 500 may change depending on the characteristics of the particular care provider viewing the GUI 500—e.g., the GUI 500 may display different information for an ECG tech than a primary care physician.

As shown, each health event 515 includes a priority 520, time stamp 525, and corresponding patient name 530. Further, event queue 505 includes a header 512 that permits the care provider viewing the GUI 500 to select how the health events 515 are ordered—e.g., by type, priority 520, time stamp 525, or patient name 530. The priority 520 may be assigned at a previous task in the workflow before the GUI 500 is displayed to the care provider 510. The priority 520 may correspond to the severity of the health event. For example, if the event manager determines that an ECG graph indicates the patient is experiencing a cardiac failure (e.g., a heart attack), the manager assigns a higher priority than a cardiac event where the ECG graph the heart rhythm is irregular but is not causing damage to the heart. In other embodiments, the age of the patient or the number of other health events associated with the patient may also be used to assign a priority 520 to the health event 515. Further, in one embodiment, the priority assigned to the health events may change dynamically.

The time stamp 525 corresponds to the time when the health event 515 was placed in the event queue. In other examples, the time stamp 525 may indicate how long the health event 515 has been in the queue 505. In one embodiment, the UI manager may color code the health events 515 to indicate priority 520 or the length of time the health event 515 has stayed in the queue 505. For example, as the priority 520 or the length of time in the queue 505 increases, the UI manager may change the color correspondingly—e.g., from green to yellow to red. In this manner, the care provider 510 can quickly identify urgent or stale health events 515 that have not yet been handled.

In one embodiment, the event queue 505 may be shared by a group of care providers 510 rather than only one provider 510. For example, different care providers 510 may view the same event queue 505 using different computing devices. As one of the care providers 510 selects a health event 515, that health event 515, the UI manager removes that event 515 from the GUIs being viewed by the other care providers 510. For example, the event queue 505 may be monitored by a group of nurses. Once a nurse selects an event 515 to handle, the UI manager updates the GUIs viewed by the other nurses in the group so that they no longer view the health event. Alternatively, instead of removing the health event from the queue 505, the GUI 500 may be updated to mark the selected event 515 as being assigned to the care provider who was chosen to handle the health event rather than unassigned.

The care provider 510 may use any input means to select one of the health events 515. As shown, the provider 510 can use a trackball, mouse, or touch pad to control a cursor 535 to select one of the health events 515. Alternatively, the input means may be a touch screen integrated into the display screen displaying GUI 500. The care provider 510 can touch the display screen to select one of the health events 515.

In one embodiment, the event queue 505 is divided into different portions such as an assigned portion for events that have been assigned to a care provider, unassigned portion for events not yet assigned to a care provider, a parked portion for events that a care provider has set aside to revisit later, and an escalated portion for the events that have been reviewed by a first care provider (e.g., a technician) but are now being submitted for review by a second care provider (e.g., a physician).

FIG. 5B illustrates a GUI 550 with an event queue 555 grouping health events 570 according to the patient associated with the health events 570. Instead of listing the health events as shown in GUI 500, the UI manager groups the health events 570 for each patient name 560. By activating the buttons 565, the care provider 510 can expand (or hide) the health events 570 for each patient. As shown, event queue 555 includes two health events 570 under patient name 560A but only one health event 570 under patient name 560B. As above, the health events 570 include a priority 575 and time stamp 580 indicating when the health events were placed in the queue 555 or how long the events 570 have been in the queue 555.

The arrangement in GUI 550 may be preferred when the care provider 510 viewing the GUI 550 is a physician or a nurse who is responsible for multiple patients. By using buttons 565, the care provider 510 can see all the events 570 for a patient and process them as a batch. Although FIG. 5B illustrates grouping the health events 570 by patient name 560, in other embodiments, the UI manager can group the health events 570 by priority 575 (e.g., high, medium, and low priority groups) or time stamp (e.g., groups for the health events that are in the queue 555 greater than five minutes, between 1-5 minutes, and less than one minute).

Returning to flow diagram 400, at step 410, the portal receives a selection from the user. In one embodiment, the selection may be made using a cursor, touch sensor, trackball, or other I/O device. For example, the user may click on a health event displayed in the first GUI. Alternatively, the portal may monitor the amount of time the cursor hovers over a particular health event in the GUI. If the time exceeds a threshold, the portal determines that the user has selected the health event.

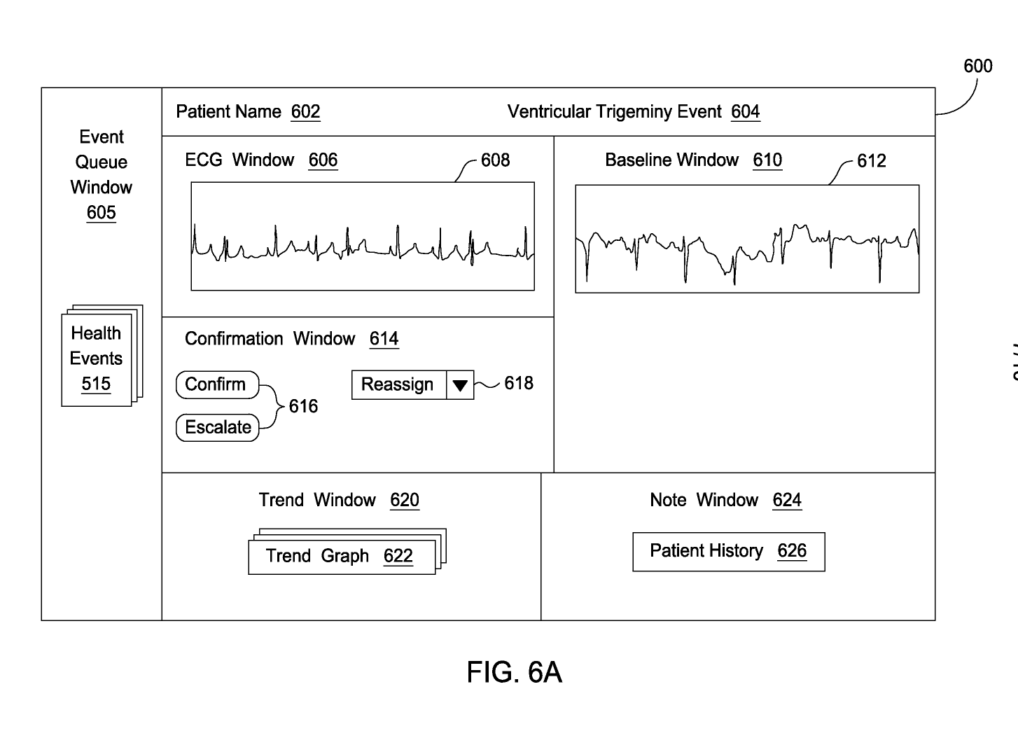

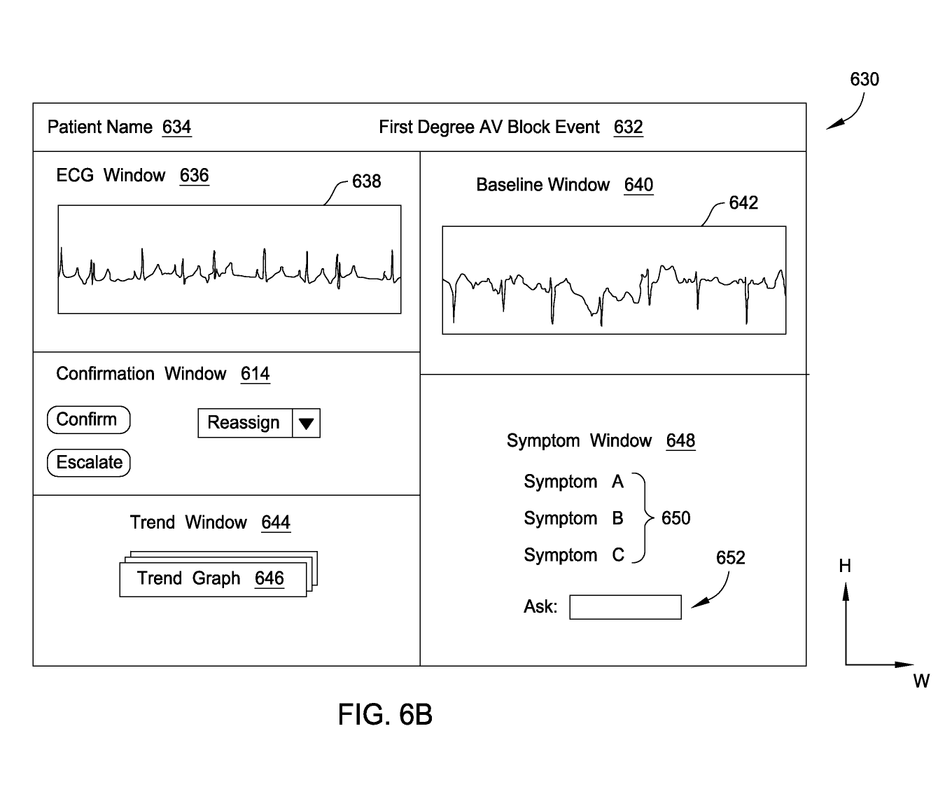

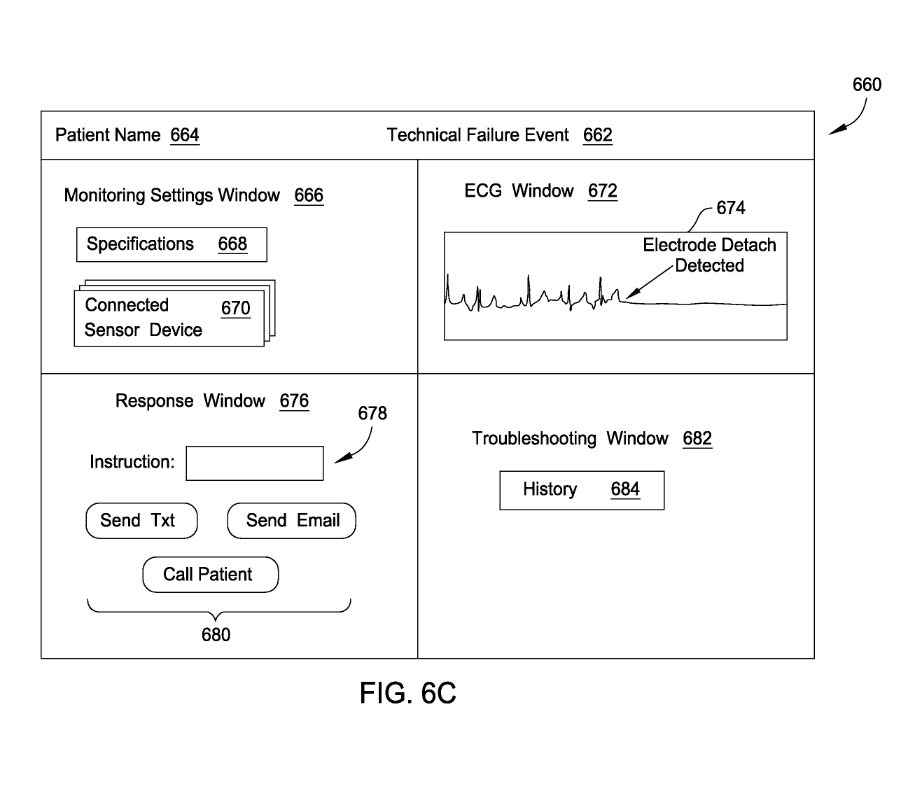

At step 415, the UI manager displays a second UI customized for the health event that was selected at step 410. Examples of GUIs that are customized for different types of health events are shown in FIGS. 6A-6C. As shown, GUI 600 in FIG. 6A includes a patient name 602 as well the type of health event 604—i.e., a ventricular trigeminy event. The GUI 600 displays information corresponding to the event 604 as well as the patient 602 in a set of defined windows. Although the windows are shown as being contiguous, in other examples there may be a spacing between the windows. In one embodiment, the windows have a fixed spatial relationship such that the care provider cannot rearrange the position of the windows. Alternatively, the windows may be selectable, and a care provider can move one window to overlap another or resize the dimensions of the windows. For example, the UI manager may use a default layout to generate a GUI for each event type which a care provider can then change to suit their particular preferences. For example, a care provider may prefer that certain windows be arranged side-by-side or that one window should take up a greater portion of the screen. After adjusting the windows, the UI manager may save the user-customized arrangement so that the next time the care provider selects a health event of the same type, the user-customized layout is used instead of the default layout.

The UI manager may permit the care provider to customize the type of windows displayed in the GUI 600 for the different health events. As shown here, GUI 600 includes an event queue window 605, ECG window 606, baseline window 610, confirmation window 614, trend window 620, and note window 624. As discussed below, the event 604 includes windows that display different information than the events displayed in FIGS. 6B and 6C. That is, the number of windows in the GUI or the information displayed in the windows (i.e., a window type) may differ depending on the type of the health event. In one embodiment, the UI manager permits the care provider to change the number of windows and the windows types displayed when the health event 604 is selected. For example, the care provider may replace the baseline window 610 with a window that displays the current medications prescribed to the patient. The UI manager may save the preferences of the care provider and use these preferences to display the GUI 600 instead of a default layout the next time the care provider selects the same type of health event 604. In this manner, the UI manager provides a flexible display environment that can be customized based on the event type as well as the care provider's personal preferences.

The event queue window 605 displays the health events 515 shown in the event queue 505 of GUI 500. For example, once the care provider selects a health event from GUI 500, the UI manager may cause GUI 500 to slide over to the right to make room in the display screen to display GUI 600. As shown here, a portion or a representation of the first GUI may remain displayed on the display screen as window 605 after the care provider selects a health event and the second GUI 600 is displayed. When doing so, the UI manager may abbreviate or otherwise reduce the information displayed in GUI 500 to fit within the event queue window 605. For example, GUI 600 includes only the names of the health events 515 but not the priority, time stamp, or patient name data shown in FIGS. 5A and 5B. The care provider can then select the health events 515 in the window 605 in order to switch to a different health event—i.e., change the GUI to no longer display information about the ventricular trigeminy event 604. Thus, event queue window 605 may provide the care provider with an easy interface for switching between events in the event queue.

In another example, the second GUI (e.g., GUI 600) may overlay the first GUI (e.g., GUI 500). For example, the first GUI may include a thumbnail or a preview area in a health event which, when selected, causes a pop-up window to appear containing the second GUI. For example, the second GUI may include only an ECG chart (e.g., graph 608) that corresponds to the health event listed in the first GUI. Thus, the first GUI may still be viewable to the user around the periphery of the second GUI. However, in other embodiments, the first GUI (or the information displayed in the GUI) may be removed completely from the screen—i.e., GUI 600 does not include the event queue window 605.

As shown in GUI 600, the care provider can use an ECG graph 608 in window 606 to determine whether the patient has an irregular heart rhythm. In some embodiments, graph 608 may include a selector bar that permits the care provider to scroll the graph 608 to see different time periods rather than the one shown when GUI 600 is first displayed. For example, the time period for graph 608 may be the previous two minutes of heart activity for the patient. To confirm that the heart activity is irregular, the care provider may increase the time range of the graph 608, assuming the data was already sent to the workflow server from the sensor devices. If not, the event manager may transmit a request to the sensor devices to send the additional heart activity.

In one embodiment, the UI manager may provide annotations on the graph 608 to indicate to the care provider where the event manager determined the patient is exhibiting an irregular heartbeat. For example, the UI manager may highlight a portion of the graph 608 or provide arrows pointing to particular sections of irregular heartbeat.

The baseline window 610 includes a baseline graph 612 that the care provider can use to evaluate the ECG graph 608. That is, the baseline graph 612 provides additional context to the care provider to determine if the heart activity is irregular. Like chart 608, the baseline graph 612 may include selection elements that permit the care provider to change the time period reflected in graph 612.

The confirmation window 614 includes action buttons 616 and dropdown box 618. The action buttons 616 permit the care provider to give instructions to the event manager which affects how the health event 604 proceeds through the workflow. For example, the care provider may select the confirm button when she agrees that the ECG graph 608 illustrates an irregular heartbeat. Stated differently, selecting the confirm button indicates that the event manager properly characterized the heartbeat as a ventricular trigeminy event. Alternatively, the care provider may select the escalate button which instructs the event manager to forward the event 604 to a different queue which may be associated with a different care provider. For example, the care provider may want to get a second opinion before deciding if the event 604 was accurately diagnosed or forward the event 604 to the patient's physician.

In one embodiment, the workflow, or more specifically, a task in the workflow, defines the set of action buttons which is then added to the UI by the UI manager. Thus, each unique workflow or task/queue within the workflow can customize the actions listed in the UI. For example, the tasks may have different action sets based on the state of the workflow, the event, etc.

The care provider may use the drop down box to reassign the event to a different event type. For example, the care provider may determine the event manager and event classifier incorrectly determined the event 604 is a ventricular trigeminy event when it actually is a different cardiac event type. In response to receiving this care provider action, the event manager may reintroduce the event into the beginning of the workflow and use the new classification to traverse the tasks. For example, referring to FIG. 4, the event classifier 112 may have previously forwarded the event 604 to task 114A after determining it was a ventricular trigeminy event. Instead, with the new classification provided by the care provider, the event manager may reintroduce the health event 604 onto a task corresponding to the new classification—e.g., task 114B or 114C. Thus, by using the buttons 616 and the drop down menu 618, the event manager is able to receive user input which is used to process the health event in the workflow. Although not shown, the confirmation window 614 may include other buttons that correspond to different actions such as storing the event 604, discarding the event 604, or instructing the event manager to delay processing the event until additional sensor data is received.

The trend window 620 displays trend graphs 622 which may provide a more long-term picture of the health of the patient. For example, if the care provider cannot determine if the specific time frame in the ECG graph 608 indicates an irregular heart rhythm, the provider may use the trend graphs 622 to determine if the occurrence of ventricular trigeminy events for the patient have increased. If the event classifier has identified the events more frequently, the care provider may determine to escalate the event rather than ignore it.

The note window 624 provides a window where the care provider can view and add notes to a patient history 626. The history 626 may include notes provided by multiple care providers (e.g., nurses, technicians, physicians) or may be previous notes made by the particular care provider who is viewing GUI 600.

FIG. 6B illustrates a GUI 630 for a health event 632. As shown, GUI 630 has a different window arrangement than GUI 600. Although GUI 630 also includes an ECG window 636, baseline window 640, confirmation window 614, and trend window 644 like GUI 600, the windows here have different dimensions (e.g., different heights and widths) when displayed on a same size display screen. For example, the width (W) of the ECG window 636 in GUI 630 may be smaller than the ECG window 606 in GUI 600, while the height (H) of the ECG windows may be the same. The other windows in GUI 630 may have different or the same dimensions as the windows in GUI 600. That is, the default arrangement of the health event 632 in FIG. 6B (i.e., a first degree AV block event) sizes the windows differently than the default arrangement of GUI 600. As above, in one embodiment, this default arrangement may be altered by the care provider such that the location and the dimensions of the windows can be changed.

GUI 630 also includes a different type of window than GUI 600. Instead of including a note window that displays patient history, GUI 630 contains a symptom window 648 that provides a list 650 of symptoms exhibited by the patient 634. In addition, the symptom window 648 includes a text box 652 (e.g., an input element) that permits the care provider to submit a question to the patient. For example, the care provider may want to know if the patient exhibits a specific symptom not included in the list 650. Once the care provider submits the query using text box 652, the event manager transmits the query to the patient's mobile device which can be used to record and return the patient's response. In one embodiment, once the care provider submits the question, the UI manager may remove GUI 630 from the display screen and permit the care provider to select a different health event. In this manner, the care provider is able to process other health events while waiting for the patient to respond. Once the response is received, the UI manager may add the event back into the event queue (e.g., GUI 500 in FIG. 5A) or display a pop-up box over the currently displayed GUI letting the care provider know that the patient has responded. The care provider can instruct the UI manager to again display GUI 630 and use the response to decide what action to take—e.g., what button to activate in the confirmation window 614.

In addition to windows having different dimensions, the information displayed within the windows may be sized differently relative to GUI 600. For example, ECG window 636 includes an ECG chart 638 which may have a smaller width or height than the ECG chart 608 in GUI 600 or the baseline chart 642 may have different dimensions than baseline chart 612 in GUI 600. Moreover, the trend graphs 646 displayed in the trend window 644 may display different trend information than the trend graphs 622 in GUI 600. In this manner, the different health events correspond to different GUIs which can have different arrangements of windows, different types of windows, different sized charts, and the like.

FIG. 6C illustrates a GUI 660 for a maintenance event 662 (i.e., a technical failure event) for a sensor device measuring biometric data for a patient 664. The GUI 660 includes a monitoring settings window 666, ECG window 672, response window 676 and troubleshooting window 682. As above, the UI manager may arrange these windows according to a default layout which the care provider can alter by moving the windows or changing their dimensions.

The monitoring settings window 666 includes a description of the mobile device used by the patient. Specifically, the window 666 includes specifications 668 of the mobile device such as the type of device (tablet or mobile phone), operating system, the version of the monitoring application executing on the mobile device, and the like. The window 666 also includes a list of the sensor devices 670 connected to the mobile device. In this embodiment, the mobile device relays the biometric data measured by the connected sensor devices 670 to the workflow server for processing. The list of connected sensor devices 670 may also include a status of these devices such as whether the devices are working properly, have a low battery, or are unavailable.

The ECG window 672 includes an ECG graph 674 which illustrates a time period where the mobile device stops receiving heart activity from a connected sensor device 670. As shown, the ECG graph 674 indicates a flat line for the heart rate for about half of the graph 674. Because the heart activity was normal before this time, the event manager or the care provider may have previously determined that the ECG graph 674 is not an indicator of a cardiac event (e.g., a heart attack) but rather a failure in the sensor device (e.g., an electrode is unplugged).

The troubleshooting window 682 includes history 684 of previous failures associated with the patient 664, mobile device, or the connected sensor devices 670. For example, when dealing with other technical failures, a care provider (e.g., an IT technician) may write in the history 684 the problem and solution. When receiving the current maintenance event 662, the care provider can use history 684 to determine what action to take. For example, the care provider may follow the same procedure a previous technician followed when handling a similar maintenance event or try a different approach if the problem is reoccurring.

Response window 676 includes a text box 678 and action buttons 680. Using the text box 678, the care provider can send instructions to the patient for troubleshooting or solving the maintenance event. For example, the care provider may instruct the patient to see if the heart activity sensor has a loose electrode or a depleted battery. The buttons 680 provide the care provider with different options for communicating with the patient such as sending a text message, email, or calling the patient.

Returning to flow diagram 400, at step 420, the event manager receives the action from the care provider using the I/O features in the second UI. For example, the care provider may activate a button or drop down menu that may confirm the diagnosis, escalate the event, instruct the event manager to store or ignore the event, provide a query or instruction to the patient, and the like. Although the GUIs shown in FIGS. 6A-6C include I/O features permitting the care provider to perform an action, in other embodiments, the care provider may use a different application or communication device (e.g., a telephone) to perform the action.

In one embodiment, the event manager performs the action requested by the care provider and adjusts the path of the event through the workflow based on the action. For example, if the care provider wants to send a text message to the patient, the event manager may forward the text message to the patient's mobile device and place the event in a queue until the patient responds. Or if the care provider determines the health event was mistakenly classified, the event manager forwards the event to the proper task or queue in the workflow. In this manner, the event manager uses the input received from the care provider to navigate the event through the workflow.

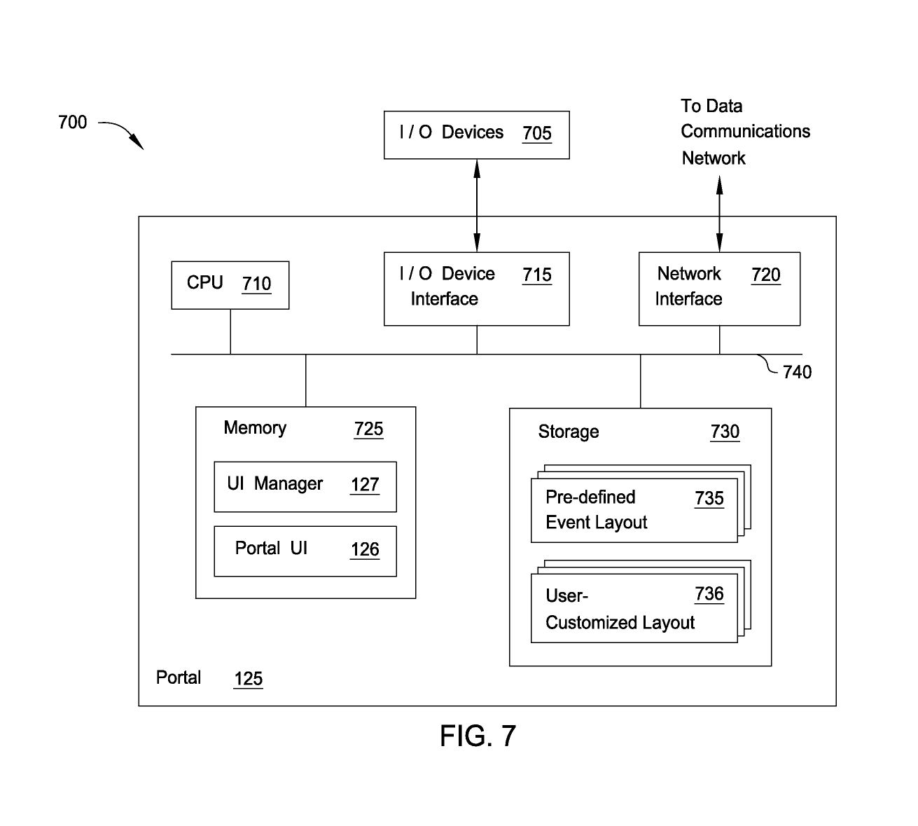

FIG. 7 illustrates the portal 125 for displaying customized GUIs in a computing system 700, according to one embodiment. For example, the computing system 700 may be part of workflow server 110 shown in FIG. 1. As shown, system 700 includes, without limitation, a central processing unit (CPU) 710, a network interface 720, a memory 725, and storage 730, each connected to a bus 740. The portal 125 may also include an I/O device interface 715 connecting I/O devices 705 (e.g., keyboard, display and mouse devices) to the portal 125. Further, in context of this disclosure, the computing elements shown in the portal 125 may correspond to a physical computing system (e.g., a system in a data center) or may be a virtual computing instance executing within a computing cloud.

CPU 710 retrieves and executes programming instructions stored in memory 725 as well as stores and retrieves application data residing in the storage 730. The bus 740 is used to transmit programming instructions and application data between CPU 710, I/O devices interface 705, storage 730, network interface 720, and memory 725. Note, CPU 710 is included to be representative of a single CPU, multiple CPUs, a single CPU having multiple processing cores, and the like. Memory 725 is generally included to be representative of a random access memory. Storage 730 may be a disk drive storage device. Although shown as a single unit, storage 730 may be a combination of fixed and/or removable storage devices, such as fixed disc drives, removable memory cards, network attached storage (NAS), or a storage area-network (SAN).

Illustratively, memory 725 includes the UI manager 127 and portal UI 126, while storage 730 includes pre-defined (or default) event layouts 735 and user-customized layouts 736. The UI manager 127 uses the pre-defined event layouts 735 or user-customized layout 736 to display the portion UI 126. For example, once a care provider has selected a health event from an event queue shown in, e.g., FIG. 5A or 5B, the UI manager 127 selects either a pre-defined layout 735 or a user-customized layout 736 corresponding to the selected health event to generate the portal UI 126 for display. If the care provider who will be viewing UI 126 has not customized or changed the pre-defined event layout 735 for the selected event, then the UI manager 127 uses the pre-defined event layout 735 to generate the UI. However, if the care provider has previously changed the arrangement, dimensions, or type of windows in the pre-defined event layout 735 to generate a corresponding customized layout 736, then the UI manager 127 uses this layout 736 instead. By storing the user-customized layout 736, the portal 125 permits each care provider to change the pre-defined layout 735 corresponding to a health event to suit her preferences. Stated differently, for each health event, the portal 125 may use a layout 736 that is customized for an individual care provider—e.g., two technicians viewing the same event may see different arrangements, dimensions, and type of windows in the portal UI 126.

One embodiment of the present disclosure is implemented as a program product for use with a computer system. The program(s) of the program product defines functions of the embodiments (including the methods described herein) and can be contained on a variety of computer-readable storage media. Examples of computer-readable storage media include (i) non-writable storage media (e.g., read-only memory devices within a computer such as CD-ROM or DVD-ROM disks readable by an optical media drive) on which information is permanently stored; (ii) writable storage media (e.g., floppy disks within a diskette drive or hard-disk drive) on which alterable information is stored. Such computer-readable storage media, when carrying computer-readable instructions that direct the functions of the present disclosure, are embodiments of the present disclosure. Other examples of media include communications media through which information is conveyed to a computer, such as through a computer or telephone network, including wireless communications networks.

In general, the routines executed to implement the embodiments of the present disclosure may be part of an operating system or a specific application, component, program, module, object, or sequence of instructions. The computer program of the present disclosure is comprised typically of a multitude of instructions that will be translated by the native computer into a machine-readable format and hence executable instructions. Also, programs are comprised of variables and data structures that either reside locally to the program or are found in memory or on storage devices. In addition, various programs described herein may be identified based upon the application for which they are implemented in a specific embodiment of the disclosure. However, it should be appreciated that any particular program nomenclature that follows is used merely for convenience, and thus the present disclosure should not be limited to use solely in any specific application identified and/or implied by such nomenclature.

As described, embodiments herein provide techniques for displaying user interfaces customized based on health event type. Advantageously, the customized user interfaces can provide information tailored to the specific health event which enables a care provider to evaluate and determine an appropriate action.

While the foregoing is directed to embodiments of the present disclosure, other and further embodiments of the disclosure may be devised without departing from the basic scope thereof, and the scope thereof is determined by the claims that follow.

What is claimed is:

1. A method, comprising:
receiving a health event for a patient derived from monitoring biometric data generated by one or more sensor devices;
generating a first graphical user interface (GUI) comprising a plurality of health events, wherein the health event is one of the plurality of health events;
upon receiving a selection of one of the plurality of health events, generating a second GUI by identifying a type of the selected health event, wherein a layout of the second GUI is dependent upon the type of the selected health event, and wherein the plurality of health events includes health events of at least two different event types.

2. The method of claim 1, further comprising:
upon receiving the selection of one of the plurality of health events, transmitting an instruction to remove the first GUI from at least a portion of a display screen viewable to a care provider, wherein the second GUI is displayed in the portion of the display screen vacated by the first GUI.

3. The method of claim 1, wherein the second UI comprises a plurality of windows, each window having a defined border and displaying different types of data associated with at least one of the patient or one or more sensor devices.

4. The method of claim 1, wherein each health event is selectable by the care provider viewing the first GUI, wherein upon receiving the selection of one of the plurality of health events from one of the plurality of care providers, the selected health event is removed from an event queue containing the plurality of health events and marked as assigned to the care provider.

5. The method of claim 4, wherein the event queue includes health events associated with a plurality of patients whose biometric data is measured using at least one respective sensor device worn by the patients.

6. The method of claim 1, further comprising:
receiving an input from a care provider viewing the second GUI via input/output (I/O) features embedded in the second GUI, the I/O features permitting the care provider to instruct a server to perform an action when processing the selected health event.

7. The method of claim 6, further comprising:
in response to receiving the input from the care provider, determining a task in a workflow to process the selected health event, the workflow comprising a plurality of interconnected tasks and queues for processing the plurality of health events.

8. A non-transitory computer-readable medium containing computer program code that, when executed by a processor, performs an operation for outputting information for display, the operation comprising:
receiving a health event for a patient derived from monitoring biometric data generated by one or more sensor devices;
generating a first GUI comprising a plurality of health events, wherein the health event is one of the plurality of health events; and
upon receiving a selection of one of the plurality of health events, generating a second GUI by identifying a type of the selected health event, wherein a layout of the second GUI is dependent upon the type of the selected health event, and wherein the plurality of health events includes health events of at least two different event types.

9. The non-transitory computer-readable medium of claim 8, the operation further comprising:
upon receiving the selection of one of the plurality of health events, transmitting an instruction to remove the first GUI from at least a portion of a display screen viewable to a care provider, wherein the second GUI is displayed in the portion of the display screen vacated by the first GUI.

10. The non-transitory computer-readable medium of claim 8, wherein the second UI comprises a plurality of windows, each window having a defined border and displaying different types of data associated with at least one of the patient or one or more sensor devices.

11. The non-transitory computer-readable medium of claim 8, wherein each health event is selectable by the care provider viewing the first GUI, wherein upon receiving the selection of one of the plurality of health events from one of the plurality of care providers, the selected health event is removed from an event queue containing the plurality of health events and marked as assigned to the care provider.

12. The non-transitory computer-readable medium of claim 11, wherein the event queue includes health events associated with a plurality of patients whose biometric data is measured using at least one respective sensor device.

13. The non-transitory computer-readable medium of claim 8, the operation further comprising:
receiving an input from a care provider viewing the second GUI via input/output (I/O) features embedded in the second GUI, the I/O features permitting the care provider to instruct a server to perform an action when processing the selected health event.

14. The non-transitory computer-readable medium of claim 13, the operation further comprising:
in response to receiving the input from the care provider, determining a task in a workflow to process the selected health event, the workflow comprising a plurality of interconnected tasks and queues for processing the plurality of health events.

15. A system, comprising:
at least one sensor device to collect biometric data associated with a patient; and
a portal configured to:
receive a health event for the patient derived from the biometric data,
generate a first GUI comprising a plurality of health events, wherein the health event is one of the plurality of health events,
upon receiving a selection of one of the plurality of health events,
generate a second GUI by identifying a type of the selected health event, wherein a layout of the second GUI is dependent upon the type of the selected health event, and wherein the plurality of health events includes health events of at least two different event types.

16. The system of claim 15, further comprising a display device configured to display the first and second GUI using a display screen, wherein the portal is configured to:
upon receiving the selection of one of the plurality of health events, transmit an instruction to the display device to remove the first GUI from at least a portion of the display screen viewable to a care provider and display the second GUI in the portion of the display screen vacated by the first GUI.

17. The system of claim 15, wherein the second UI comprises a plurality of windows, each window having a defined border and displaying different types of data associated with at least one of the patient or the sensor device.

18. The system of claim 15, further comprising a workflow comprising a plurality of interconnected tasks and queues used for processing the plurality of health events.

19. The system of claim 18, wherein a workflow server routes the health event to a task in the workflow for further processing based on an input provided by a care provider viewing the second GUI via I/O features embedded in the second GUI.

20. The system of claim 18, further comprising a mobile device associated with the patient configured to receive the biometric data from the sensor device and forward the biometric data to the workflow.
